(19) United States
(12) Patent Application Publication
(10) Pub. No.: US 2025/0195884 A1
(43) Pub. Date: Jun. 19, 2025

(54) PHYSIOLOGICAL SENSING AND/OR STIMULATION PROBE AND METHOD THEREOF

(71) Applicant: INDUSTRIAL TECHNOLOGY RESEARCH INSTITUTE, Hsinchu (TW)

(72) Inventors: Jo-Ping LEE, Hsinchu City (TW); Chung-Hsin SU, Hsinchu City (TW); Kun-Ta WU, Nantou City (TW)

(73) Assignee: INDUSTRIAL TECHNOLOGY RESEARCH INSTITUTE, Hsinchu (TW)

(21) Appl. No.: 18/396,361

(22) Filed: Dec. 26, 2023

(30) Foreign Application Priority Data
Dec. 15, 2023 (TW) 112149083

(51) Int. Cl. A61N 1/36; A61N 1/04; G16H 70/00

(52) U.S. Cl. CPC A61N 1/36031; A61N 1/0472; G16H 70/00

(57) ABSTRACT

A physiological sensing and/or stimulation probe and method. The physiological sensing and/or stimulation probe includes a probe body, a first electrode, a second electrode and an adjusting unit. The first electrode is disposed on the probe body. The second electrode is spaced apart from the first electrode and has an outer surface facing away from the first electrode. The second electrode is movably disposed on the probe body via the adjusting unit so that an angle between a normal direction of the outer surface of the second electrode and a central line of the probe body is adjustable, thereby allowing an electrical field generated between the first electrode and the second electrode to be adjustable.

### CROSS-REFERENCE TO RELATED APPLICATIONS

All related applications are incorporated by reference. The present application is based on, and claims priority from, Taiwan (International) application No. 112149083 filed on Dec. 15, 2023, the disclosure of which is hereby incorporated by reference herein in its entirety.

### TECHNICAL FIELD

The disclosure relates to a physiological sensing and/or stimulation probe and method.

### BACKGROUND

In general, in order to cure the movement disorder of the patient, a probe is inserted into the brain to apply an electrical stimulation thereto. When the scope where the electrical stimulation is applied is not accurately controlled, the electrical stimulation may cause side effects in the cells of the brain. Such side effects may be examined via a preliminary test, including the examination on muscle tone of the patient and the examination on the brainwave response to light. Thus, a probe causing less side effects is required to be developed.

### SUMMARY

One embodiment of the disclosure provides a physiological sensing and/or stimulation probe including a probe body, a first electrode, a second electrode and an adjusting unit. The first electrode is disposed on the probe body. The second electrode is spaced apart from the first electrode and has an outer surface facing away from the first electrode. The second electrode is movably disposed on the probe body via the adjusting unit so that an angle between a normal direction of the outer surface of the second electrode and a central line of the probe body is adjustable, thereby allowing an electrical field generated between the first electrode and the second electrode to be adjustable.

Another embodiment of the disclosure provides a physiological sensing and/or stimulation method including: controlling an adjusting unit of an physiological sensing and/or stimulation probe by a controlling device, so as to adjust an angle between a normal direction of an outer surface of a second electrode of the physiological sensing and/or stimulation probe facing away from a first electrode and a central line of a probe body of the physiological sensing and/or stimulation probe to a plurality of test angles, thereby obtaining at least one physiological sensing data on at least one test plane in an affected part where the physiological sensing and/or stimulation probe is inserted.

Still another embodiment of the disclosure provides a physiological sensing and/or stimulation method including: generating a comparison result by comparing at least one physiological sensing data with a physiological sensing reference data via a controlling device; and controlling an adjusting unit of a physiological sensing and/or stimulation probe, so as to adjust an angle between a normal direction of an outer surface of a second electrode of the physiological sensing and/or stimulation probe facing away from a first electrode and a central line of a probe body of the physiological sensing and/or stimulation probe to a stimulation applying angle according to the comparison result via the controlling device, and controlling the physiological sensing and/or stimulation probe to apply an electrical stimulation to the affect part via the controlling device.

### BRIEF DESCRIPTION OF THE DRAWINGS

The present disclosure will become better understood from the detailed description given herein below and the accompanying drawings which are given by way of illustration only and thus are not intending to limit the present disclosure and wherein:

FIG. 1 is a partially enlarged perspective view of a physiological sensing and/or stimulation system according to a first embodiment of the disclosure.

FIG. 2 is a partially enlarged side view of a physiological sensing and/or stimulation probe in FIG. 1.

FIG. 3 is a partially enlarged side view showing that a second electrode of the physiological sensing and/or stimulation probe in FIG. 1 swing to be at a first angle.

FIG. 4 is a partially enlarged side view showing that a second electrode of the physiological sensing and/or stimulation probe in FIG. 1 swing to be at a second angle.

FIG. 5 is a partially enlarged side view of a physiological sensing and/or stimulation probe according to a second embodiment of the disclosure.

FIG. 6 is a flow chart of a physiological sensing and/or stimulation method using the physiological sensing and/or stimulation probe in FIG. 1.

FIG. 7 schematically shows the physiological sensing and/or stimulation method in FIG. 6.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

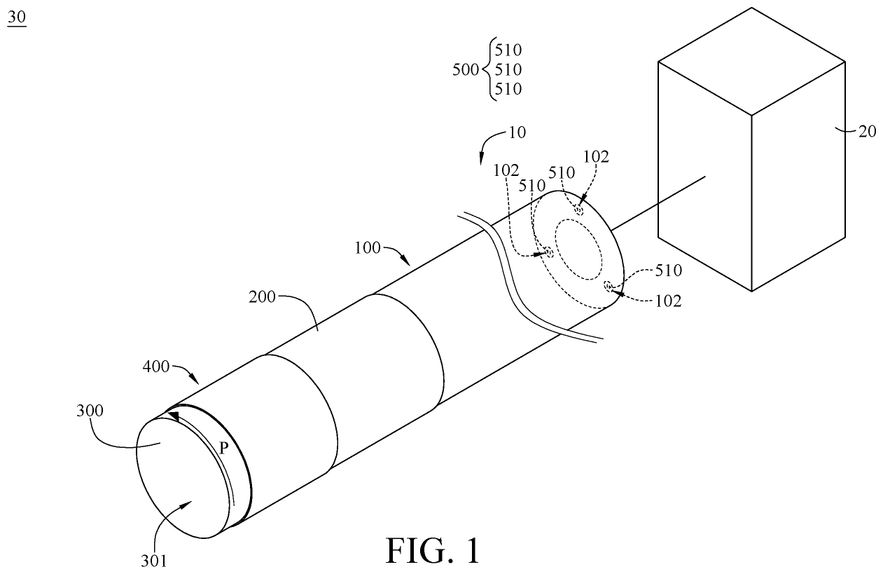

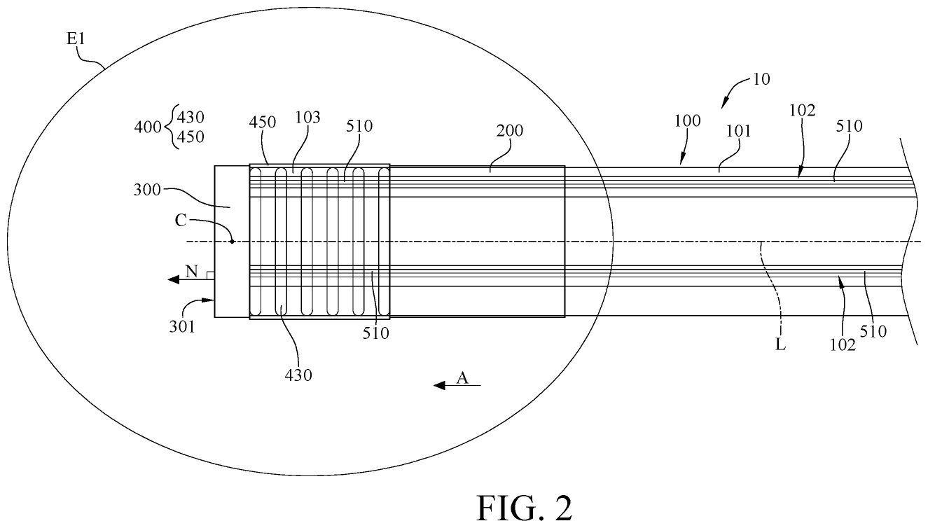

Please refer to FIGS. 1 and 2. FIG. 1 is a partially enlarged perspective view of a physiological sensing and/or stimulation system according to a first embodiment of the disclosure. FIG. 2 is a partially enlarged side view of a physiological sensing and/or stimulation probe in FIG. 1. In order to highlight the technical features of the disclosure, the components that are blocked are shown by solid lines.

In this embodiment, the physiological sensing and/or stimulation system 30 includes a physiological sensing and/or stimulation probe 10 and a controlling device 20. The physiological sensing and/or stimulation probe 10 includes a probe body 100, a first electrode 200, a second electrode 300, a flexible unit 400 and an adjusting unit 500.

The probe body 100 is made of, for example, an insulative material with biocompatibility, biological stability and flexibility. The probe body 100 includes a hollow pipe part 101 extending along an axial direction A and a plurality of mounting channels 102 that are spaced apart from one another and penetrate through the hollow pipe part 101 along the axial direction A.

The first electrode 200 is disposed on the probe body 100, and is located adjacent to a free end 103 of the probe body 100. In this embodiment, the first electrode 200 is in, for example, a ring shape and surrounds the probe body 100.

The second electrode 300 is spaced apart from the first electrode 200, and has an outer surface 301 facing away from the first electrode 200. In this embodiment, the second electrode 300 is in, for example, a sheet shape, and is disposed on the free end 103 of the probe body 100. In this embodiment, a central line L of the probe body 100 passes through a geometric center C of the second electrode 300. In addition, in this embodiment, the second electrode 300 is, for example, a planar conductor, but the disclosure is not limited thereto. In other embodiments, the second electrode may be a curved conductor. The first electrode 200 and the second electrode 300 may, for example, be electrically connected to the controlling device 20 via one or more wires, respectively. In order to prevent the wires from obscuring the main point of the disclosure by being shown in the drawings, the wires are not shown in the drawings. The first electrode 200 and the second electrode 300 are configured to sense physiological signals and/or configured to provide an electrical stimulation via an electrical field defined by generating directional current or voltage output, which will be described in detail in the descriptions regarding the physiological sensing and/or stimulation method below.

The adjusting unit 500 is configured to allow the second electrode 300 to swing relative to the first electrode 200, thereby adjusting an angle between the normal direction N of the outer surface 301 of the second electrode 300 and the central line L of the probe body 100. The adjusting unit 500 includes a plurality of adjusting components 510. The adjusting components 510 are movably disposed in the corresponding mounting channels 102 of the probe body 100 along the axial direction A of the probe body 100. That is, the controlling device 20 is able to control a driving component (not shown) to pull or move the adjusting components 510, so as to allow the adjusting components 510 to be moved in the mounting channels 102. Alternatively, the adjusting components 510 may be manually pulled to be moved in the mounting channels 102. Ends of the adjusting components 510 are fixed to the second electrode 300, spaced apart from one another, and arranged along a circumferential direction P of the second electrode 300.

In this embodiment, there are, for example, three adjusting components 510 to obtain physiological sensing data of different positions on any test plane and/or to provide the electrical stimulation on a target position. However, the disclosure is not limited to the number of the adjusting components 510. In other embodiments, there may be four or more adjusting components. Alternatively, in still other embodiments, if a stimulation applying angle is not required to be optimized by building two-dimensional or three-dimensional physiological sensing data model, there may be two adjusting components. In this embodiment, the number of the mounting channels 102 of the probe body 100 is equal to the number of the adjusting components 510. However, the disclosure is not limited thereto. In other embodiments, as long as the adjusting components are spaced apart from one another and arranged along the circumferential direction of the second electrode, the number of the mounting channels of the probe body may be correspondingly adjusted to be different from the number of the adjusting components. Additionally, in this embodiment, the adjusting components 510 of the adjusting unit 500 are, for example, flexible threads, but the disclosure is not limited to the configuration of the adjusting components 510. In other embodiments, the adjusting components may have any configuration that allows the angle between the second electrode and the first electrode to be adjustable.

The flexible unit 400 is configured to provide an auxiliary supporting force when the second electrode 300 swings relative to the first electrode 200. The flexible unit 400 surrounds the probe body 100, and includes a flexible component 430 and a flexible sleeve 450. The flexible component 430 is covered on the free end 103 of the probe body 100, and is located between the first electrode 200 and the second electrode 300. In this embodiment, the flexible component 430 is, for example, a linear spring made of metal or plastic. The flexible sleeve 450 is configured to allow the flexible component 430 to be located between the first electrode 200 and the second electrode 300. In this embodiment, the flexible sleeve 450 is made of, for example, an insulative material with biocompatibility, biological stability and flexibility, such as polyethylene terephthalate (PET), Polytetrafluoroethylene (PTFE), Ethylene tetrafluoroethylene (ETFE), and Fluorinated ethylene propylene (FEP). Moreover, the flexible sleeve 450 is covered on the flexible component 430 and the free end 103 of the probe body 100. The flexible sleeve 450, for example, avoids the flexible component 430 from damaging the affected part due to the direct contact therebetween.

Note that the disclosure is not limited to the coverage of the flexible sleeve 450. In other embodiments, the flexible sleeve may be covered on the flexible component, the free end and a part of the hollow pipe part.

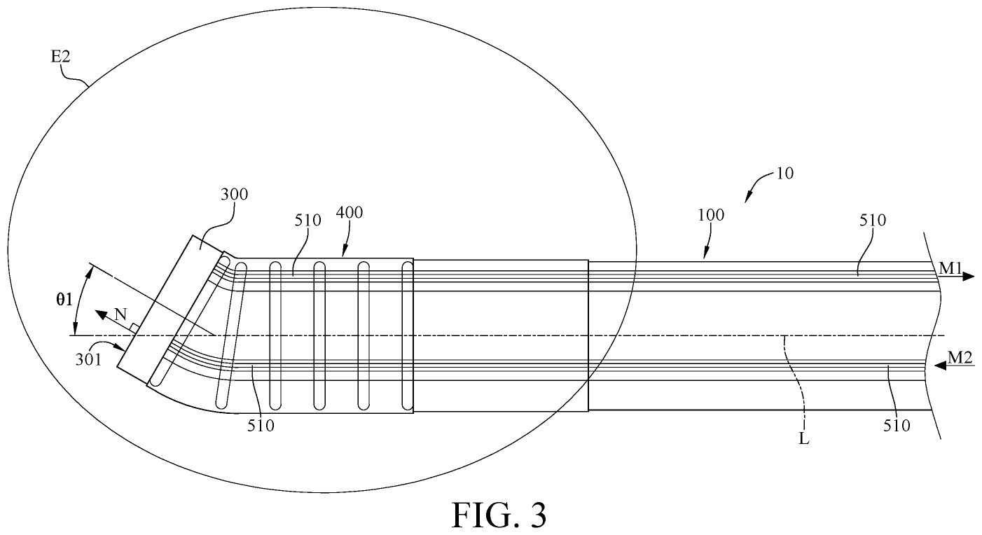

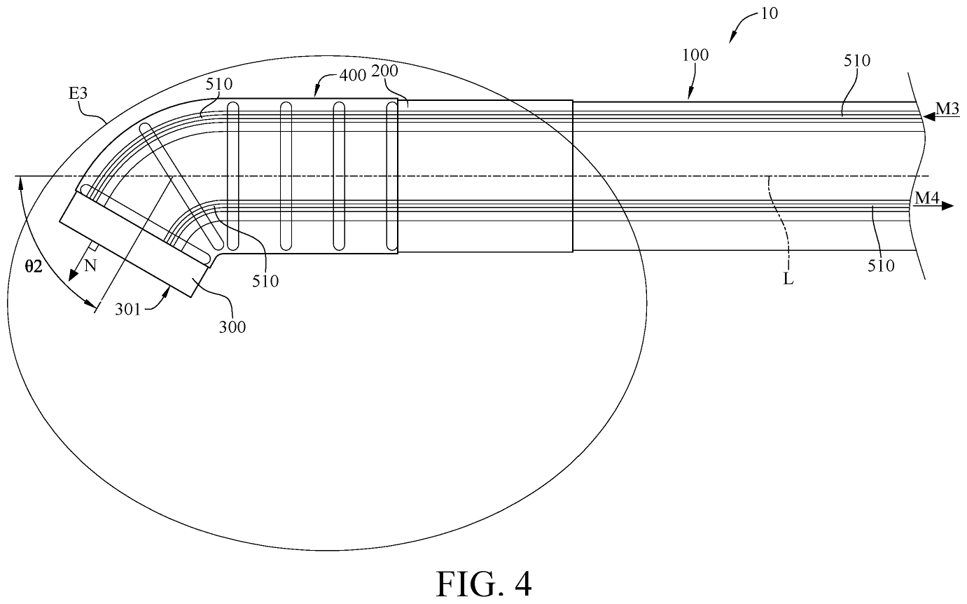

Please refer to FIGS. 2 to 4. FIG. 3 is a partially enlarged side view showing that a second electrode of the physiological sensing and/or stimulation probe in FIG. 1 swing to be at a first angle. FIG. 4 is a partially enlarged side view showing that a second electrode of the physiological sensing and/or stimulation probe in FIG. 1 swing to be at a second angle. The second electrode 300 is swingably disposed on the free end 103 of the probe body 100. Thus, the angle between the normal direction N of the outer surface 301 of the second electrode 300 and the central line L of the probe body 100 is adjustable, thereby allowing an electrical field E1 generated between the first electrode 200 and the second electrode 300 to be adjustable. In this way, the direction and scope of the electrical stimulation applied by the probe 10 are adjustable. For example, the electrical field E1 generated between the first electrode 200 and the second electrode 300 may be defined by the directional current or voltage output generated thereby. Also, the angle between the normal direction N and the central line L may be changed from 0 degree as shown in FIG. 2 (i.e., the normal direction N being parallel to the central line L) to the first angle θ1 of, for example, 30 degrees as shown in FIG. 3, or to the second angle θ2 of, for example, −60 degrees as shown in FIG. 4. Specifically, in FIG. 3, the adjusting unit 500 is moved along moving directions M1 and M2 to allow the angle between the normal direction N and the central line L to be adjusted to 30 degrees. In FIG. 4, the adjusting unit 500 is moved along moving directions M3 and M4 to allow the angle between the normal direction N and the central line L to be adjusted to −60 degrees, where the moving directions M1 and M2 are opposite to the moving directions M3 and M4, respectively. Further, after the second electrode 300 swings, the electrical field E1 shown in FIG. 2 may be adjusted to an electrical field E2 shown in FIG. 3 or an electrical field E3 shown in FIG. 4.

Since the central line L of the probe body 100 passes through the geometric center C of the second electrode 300, a radial offset of the second electrode 300 from the central line L when swinging is reduced. Accordingly, the side effects are prevented from being generated in the cells around the affected part due to the radial offset of the second electrode 300.

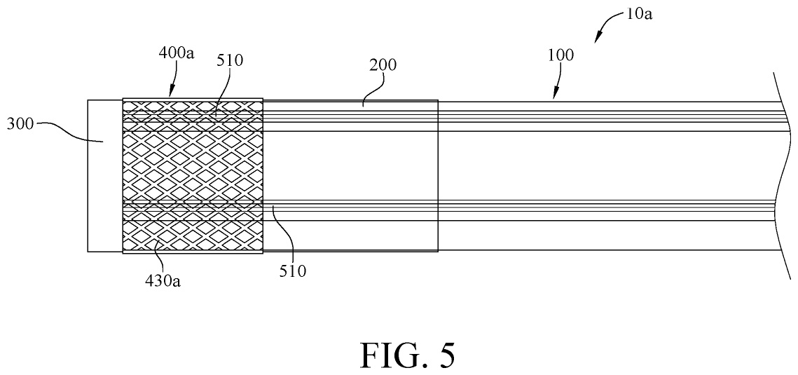

The disclosure is not limited to the configuration of the flexible component. Please refer to FIG. 5. FIG. 5 is a partially enlarged side view of a physiological sensing and/or stimulation probe according to a second embodiment of the disclosure. This embodiment uses the reference numerals and a part of the contents of the first embodiment. The same reference numerals are used to denote the same or similar elements, and the description of the same technical contents is omitted. For the description of the omitted part, reference may be made to the first embodiment, and details are not described in this embodiment. The difference between the physiological sensing and/or stimulation probe 10a of this embodiment and the physiological sensing and/or stimulation probe 10 of the first embodiment is merely in the configuration of the flexible component 430a of the flexible unit 400a. In this embodiment, the flexible component 430a may be a weave structure or a spring in a mesh shape or a strain relief. The flexible component 430a in FIG. 5 is, for example, a weave structure in a mesh shape.

First, the relationship between the controlling device 20 and the physiological sensing and/or stimulation probe 10 is described. The controlling device 20 is configured to control the adjusting components 510 of the adjusting unit 500, thereby adjusting the angle of the second electrode 300 and the electrical field generated between the first electrode 200 and the second electrode 300. In addition, the controlling device 20 is configured to apply voltage or current to the first electrode 200 and the second electrode 300 to perform physiological sensing or apply stimulation. Also, the controlling device 20 is configured to compare the data and magnitude.

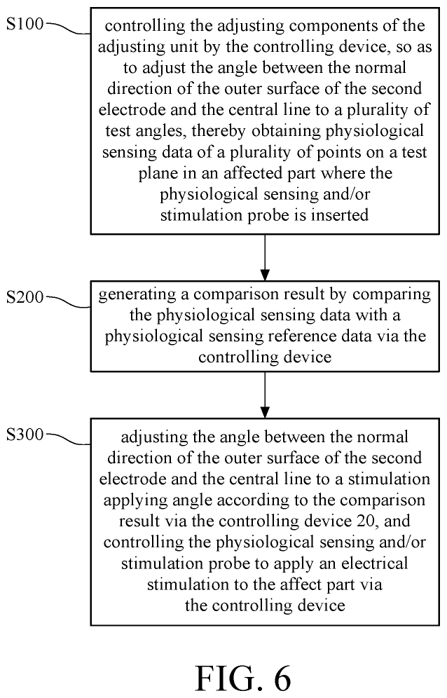

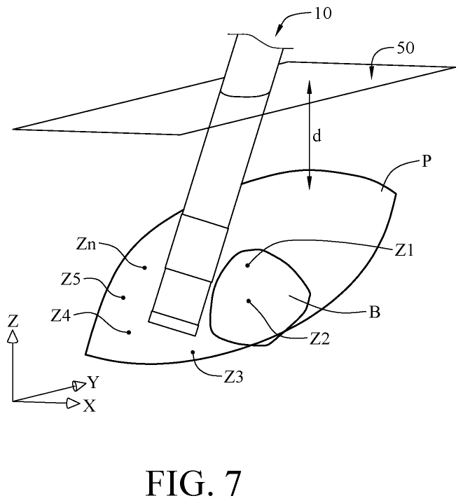

Hereinafter, a physiological sensing and/or stimulation method performed by using the physiological sensing and/or stimulation system 30 in FIG. 1 will be described. Please refer to FIGS. 2, 6 and 7. FIG. 6 is a flow chart of a physiological sensing and/or stimulation method using the physiological sensing and/or stimulation probe in FIG. 1. FIG. 7 schematically shows the physiological sensing and/or stimulation method in FIG. 6. The physiological sensing and/or stimulation method may include steps S100, S200 and S300.

First, the step S100 is performed to control the adjusting components 510 of the adjusting unit 500 by the controlling device 20, so as to adjust the angle between the normal direction N of the outer surface 301 of the second electrode 300 and the central line L to a plurality of test angles, thereby obtaining physiological sensing data of a plurality of points Z1-Zn on a test plane P in an affected part 50 where the physiological sensing and/or stimulation probe 10 is inserted. That is, in the step S100, the physiological sensing is performed by the controlling device 20. The physiological sensing data is, for example, resistance data. The affected part 50 is, for example, a brain. The test plane P is, for example, parallel to the X-Y plane and has a depth d along Z-axis direction. In this embodiment, when the physiological sensing and/or stimulation probe 10 is inserted in different test planes P, the physiological sensing and/or stimulation probe 10 has different depths d relative to the affected part 50. In other words, in this embodiment, for example, the physiological sensing and/or stimulation probe 10 adjusts the electrical field at different depths d to build a three-dimensional physiological sensing data mode.

For example, if there are two test angles, the test angles may be 0 degree and 180 degrees, respectively; if there are three test angles, the test angles may be 0 degree, 120 degrees and 240 degrees, respectively. However, the disclosure is not limited to the number of the test angles. That is, the disclosure is not limited to the times for performing physiological sensing. In other embodiments, the number of the test angles may be determined by the required times for performing physiological sensing.

Next, the step S200 is performed to generate a comparison result by comparing the physiological sensing data with a physiological sensing reference data via the controlling device 20. For example, the controlling device 20 compares the physiological sensing data with the reference magnitude of the physiological sensing reference data, thereby determining the regions that is required to allow the stimulation to be applied thereon, such as region B in FIG. 7 where the points Z1-Z2 are located. The physiological sensing reference data is from, for example, a database (not shown) electrically connected to the controlling device 20.

Next, the step S300 is performed to adjust the angle between the normal direction N of the outer surface 301 of the second electrode 300 and the central line L to a stimulation applying angle according to the comparison result via the controlling device 20, and to control the physiological sensing and/or stimulation probe 10 to apply an electrical stimulation to the affect part 50 via the controlling device 20.

In this embodiment, the three-dimensional physiological sensing data model is built according to the test planes of different depths, but the disclosure is not limited thereto. In other embodiments, a two-dimensional physiological sensing data model may be built according to a single test plane.

In this embodiment, the physiological sensing and/or stimulation method includes the step S100 for physiological sensing and the steps S200 and S300 for applying stimulation. However, the physiological sensing and/or stimulation method according to the disclosure may perform physiological sensing and/or apply stimulation. That is, in other embodiments, the physiological sensing and/or stimulation method may merely include the step S100 for physiological sensing, or may merely include the steps S200 and S300 for applying stimulation.

According to the physiological sensing and/or stimulation system and method disclosed by above embodiments, the second electrode is swingably disposed on the probe body or the first electrode, and thus the angle between the normal direction of the outer surface of the second electrode and the central line is adjustable. Therefore, the electrical field generated between the first electrode and the second electrode is adjustable. In this way, the direction and the scope of the electrical stimulation is allowed to be accurately controlled by adjusting the angle of the second electrode, thereby reducing the side effects.

In addition, the physiological sensing data on the test plane are obtained by the angle adjustment performed by at least three adjusting components, and the angle of the second electrode is adjusted according to the comparison of the physiological sensing data. In this way, the direction and the scope of the electrical stimulation is allowed to be accurately controlled, thereby reducing the side effects.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A physiological sensing and/or stimulation probe, comprising:
a probe body;
a first electrode, disposed on the probe body;
a second electrode, spaced apart from the first electrode and having an outer surface facing away from the first electrode; and
an adjusting unit, wherein the second electrode is movably disposed on the probe body via the adjusting unit so that an angle between a normal direction of the outer surface of the second electrode and a central line of the probe body is adjustable, thereby allowing an electrical field generated between the first electrode and the second electrode to be adjustable.

2. The physiological sensing and/or stimulation probe according to claim 1, further comprising a flexible unit, wherein the second electrode is movably disposed on the probe body via the flexible unit.

3. The physiological sensing and/or stimulation probe according to claim 2, wherein the flexible unit comprises a flexible component covered on the probe body and located between the first electrode and the second electrode.

4. The physiological sensing and/or stimulation probe according to claim 3, wherein the flexible unit further comprises a flexible sleeve configured to allow the flexible component to be located between the first electrode and the second electrode.

5. The physiological sensing and/or stimulation probe according to claim 1, wherein the adjusting unit comprises at least two adjusting components, the at least two adjusting components are movably disposed in the probe body along an axial direction of the probe body, ends of the at least two adjusting components are fixed to the second electrode, spaced apart from each other, and arranged along a circumferential direction of the second electrode.

6. The physiological sensing and/or stimulation probe according to claim 5, wherein the at least two adjusting components comprise three adjusting components.

7. The physiological sensing and/or stimulation probe according to claim 1, wherein the central line of the probe body passes through a geometric center of the second electrode.

8. The physiological sensing and/or stimulation probe according to claim 1, wherein the first electrode is in a ring shape and surrounds the probe body.

9. The physiological sensing and/or stimulation probe according to claim 1, wherein the probe body has at least two mounting channels, and the at least two adjusting components are movably disposed in the at least two mounting channels, respectively.

10. A physiological sensing and/or stimulation method, comprising:
controlling an adjusting unit of an physiological sensing and/or stimulation probe by a controlling device, so as to adjust an angle between a normal direction of an outer surface of a second electrode of the physiological sensing and/or stimulation probe facing away from a first electrode and a central line of a probe body of the physiological sensing and/or stimulation probe to a plurality of test angles, thereby obtaining at least one physiological sensing data on at least one test plane in an affected part where the physiological sensing and/or stimulation probe is inserted.

11. A physiological sensing and/or stimulation method, comprising:
generating a comparison result by comparing at least one physiological sensing data with a physiological sensing reference data via a controlling device; and
controlling an adjusting unit of a physiological sensing and/or stimulation probe, so as to adjust an angle between a normal direction of an outer surface of a second electrode of the physiological sensing and/or stimulation probe facing away from a first electrode and a central line of a probe body of the physiological sensing and/or stimulation probe to a stimulation applying angle according to the comparison result via the controlling device, and controlling the physiological sensing and/or stimulation probe to apply an electrical stimulation to the affect part via the controlling device.
